(19) United States
(12) Patent Application Publication
(10) Pub. No.: US 2008/0292164 A1
(43) Pub. Date: Nov. 27, 2008

(54) SYSTEM AND METHOD FOR COREGISTRATION AND ANALYSIS OF NON-CONCURRENT DIFFUSE OPTICAL AND MAGNETIC RESONANCE BREAST IMAGES

(75) Inventors: Fred S. Azar, Princeton, NJ (US); Arjun G. Yodh, Merion, PA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Corporate Research, Inc., Princeton, NJ (US)

(21) Appl. No.: 11/845,183

(22) Filed: Aug. 27, 2007

Related U.S. Application Data
(60) Provisional application No. 60/840,761, filed on Aug. 29, 2006.

(51) Int. Cl. A61B 5/055

(52) U.S. Cl. 382/131

(57) ABSTRACT

A method for joint analysis of non-concurrent magnetic resonance (MR) and diffuse optical tomography (DOT) images of the breast includes providing a digitized MR breast image volume comprising a plurality of intensities corresponding to a 3-dimensional (3D) grid of voxels, providing a digitized DOT breast dataset comprising a plurality of physiological values corresponding to a finite set of points, segmenting the breast MR image volume to separate tumorous tissue from non-tumorous tissue, registering a DOT breast dataset and the MR image volume and fusing said registered DOT and MR datasets, wherein said fused dataset is adapted for analysis.

### CROSS REFERENCE TO RELATED UNITED STATES APPLICATIONS

This application claims priority from “A Method for Joint Analysis of Non-Concurrent Magnetic Resonance Imaging and Diffuse Optical Tomography of Breast Cancer”, U.S. Provisional Application No. 60/840,761 of Azar, et al. filed Aug. 29, 2006, the contents of which are herein incorporated by reference.

### TECHNICAL FIELD

This disclosure is directed to methods for combining breast image data obtained at different times, in different geometries and by different techniques.

### DISCUSSION OF THE RELATED ART

Near-infrared (NIR) diffuse optical tomography (DOT) relies on functional processes, and provides unique measurable parameters with potential to enhance breast tumor detection sensitivity and specificity. For example, several groups have demonstrated the feasibility of breast tumor characterization based on total hemoglobin concentration, blood oxygen saturation, water and lipid concentration and scattering.

The functional information derived with DOT is complementary to structural and functional information available to conventional imaging modalities such as magnetic resonance imaging (MRI), X-ray mammography and ultrasound. Thus the combination of functional data from DOT with structural/anatomical data from other imaging modalities holds potential for enhancing tumor detection sensitivity and specificity. In order to achieve this goal of data fusion, two general approaches can be employed. The first, concurrent imaging, physically integrates the DOT system into the conventional imaging instrument. This approach derives images in the same geometry and at the same time. The second approach, non-concurrent imaging, employs optimized stand-alone DOT devices to produce 3D images that must then be combined with those of the conventional imaging modalities via software techniques. In this case the images are obtained at different times and often in different geometries.

Thus far a few DOT systems have been physically integrated into conventional imaging modalities such as MRI, X-ray mammography, and ultrasound for concurrent measurements. By doing so, however, these DOT systems are often limited by the requirements of the ‘other’ imaging modality, for example, restrictions on metallic instrumentation for MRI, hard breast compression for X-ray mammography, limited optode combinations for ultrasound (and MRI, X-Ray) and time constraints. On the other hand, among the stand-alone DOT systems available today, only a few attempts have been made to quantitatively compare DOT images of the same breast cancer patient to those of other imaging modalities obtained at different times because non-concurrent coregistration presents many challenges. It is therefore desirable to develop quantitative and systematic methods for data fusion that utilize the high-quality data and versatility of the stand-alone imaging systems.

3D-DOT/3D-MRI image registration presents several new challenges. Because registration of DOT to MR acquired non-concurrently has not been extensively studied, no standard approach is known to have been established for this procedure. DOT images have much lower anatomical resolution and contrast than MRI, and the optical reconstruction process typically uses a geometric model of the breast. An exemplary constraining geometric model of the breast is a semi-ellipsoid. Typically, the patient breast is compressed axially in the DOT imaging device and sagitally in the MRI machine, and, of course, the breast is a highly deformable organ.

Automatic image registration is a useful component in medical imaging systems. The basic goal of intensity based image registration techniques is to align anatomical structures in different modalities. This is done through an optimization process, which assesses image similarity and iteratively changes the transformation of one image with respect to the other, until an optimal alignment is found. Computation speed can dictate applicability of the technology in practice. Although feature based methods are computationally more efficient, they are dependant on the quality of the extracted features from the images.

In intensity based registration, volumes are directly aligned by iteratively computing a volumetric similarity measure based on the voxel intensities. Since the amount of computation per iteration is high, the overall registration process is slow. In the cases where Mutual Information (MI) is used, sparse sampling of volume intensity could reduce the computational complexity while compromising the accuracy.

### SUMMARY OF THE INVENTION

Exemplary embodiments of the invention as described herein generally include methods and systems for fusing and jointly analyzing multimodal optical imaging data with X-Ray tomosynthesis and MR images of the breast. A method and system according to an embodiment of the invention integrates advanced multimodal registration and segmentation algorithm. Coregistration combines structural and functional data from multiple modalities, while segmentation and fusion will also enable a priori structural information derived from MRI to be incorporated into the DOT reconstruction algorithms. The combined MRI/DOT data set provides information in a more useful format than the sum of the individual datasets. The resulting superposed 3D tomographs facilitate tissue analyses based on structural and functional data derived from both modalities and readily permit enhancement of DOT data reconstruction using MRI-derived a priori structural information.

A method and system according to an embodiment of the invention uses a straight-forward and well-defined workflow that requires little prior user interaction, and is robust enough to handle a majority of patient cases, computationally efficient for practical applications, and yields results useful for combined MRI/DOT analysis. This system is more flexible than integrated MRI/DOT imaging systems in the system design and patient positioning and enables the independent development of a standalone DOT system without the limitations imposed by the MRI device environment.

A multi-modal registration method according to an embodiment of the invention was tested using a simulated phantom, and with actual patient data. These studies confirm that tumorous regions in a patient breast found by both imaging modalities exhibit significantly higher total hemoglobin concentration (THC) than surrounding normal tissues. The average THC in the tumorous regions is one to three standard deviations larger than the overall breast average THC for all patients. These results show that functional information on a tumor obtained from DOT data can be combined with the anatomy of that tumor derived from MRI data.

A system and method according to an embodiment of the invention can contribute to standardizing the direct comparison of the two modalities (MRI and DOT), and should have a positive impact on standardization of optical imaging technology, through establishing common data formats and processes for sharing data and software, which in turn will allow direct comparison of different modalities, validation of new versus established methods in clinical studies, development of commonly accepted standards in post-processing methods, creation of a standardized MR-DOT technology platform and, eventually, translation of research prototypes into clinical imaging systems.

According to an aspect of the invention, there is provided a method for joint analysis of non-concurrent magnetic resonance (MR) and diffuse optical tomography (DOT) images of the breast, including providing a digitized MR breast image volume comprising a plurality of intensities corresponding to a 3-dimensional (3D) grid of voxels, providing a digitized DOT breast dataset comprising a plurality of physiological values corresponding to a finite set of points, segmenting said breast MR image volume to separate tumorous tissue from non-tumorous tissue, registering said DOT breast dataset and said MR image volume, and fusing said registered DOT and MR datasets, wherein said fused dataset is adapted for analysis.

According to a further aspect of the invention, the physiological values include one or more of total hemoglobin concentration, blood oxygenation saturation, and light scattering data.

According to a further aspect of the invention, segmenting said breast MR image volume comprises selecting at least one axial, at least one coronal, and at least one sagittal slice of said MR image volume, selecting 3 different seed points in each selected slice, said seed points representative of fatty breast tissue, non-fatty breast tissue, and non-breast tissue, determining a probability that a random walker starting at an unselected point reaches one of said selected seed points, and labeling each unselected point according to the seed point with a highest probability to create a mask file, wherein each point in each slice is labeled as fatty breast tissue, non-fatty breast tissue, or non-breast tissue.

According to a further aspect of the invention, the method includes resampling said DOT dataset into a 3D volume of voxels corresponding to said MR grid of voxels.

According to a further aspect of the invention, the method includes incorporating said mask file into said DOT dataset.

According to a further aspect of the invention, registering said DOT breast dataset to said MR image volume comprises generating a 2D sagittal projection signature from said MR image and from said DOT dataset, registering said DOT sagittal signature and said MR sagittal signature, generating a 2D coronal projection signature from said MR image and from said DOT dataset, registering said DOT coronal signature and said MR coronal signature, generating a 2D axial projection signature from said MR image and from said DOT dataset, registering said DOT axial signature and said MR axial signature, wherein said 3D registration mapping is defined in terms of said 2D sagittal, coronal, and axial registrations.

According to a further aspect of the invention, the steps of generating a 2D projection signature and registering said signatures for each of said sagittal, coronal, and axial projections is repeated for a pre-determined number of iterations.

According to a further aspect of the invention, the 2D projection signatures are generated from a maximum intensity projection.

According to a further aspect of the invention, one of said DOT and MR signatures is a moving signature and the other is a fixed signature, and wherein registering a DOT signature and an MR signature comprises, initializing deformation variables for scaling said moving signature vertically and horizontally, translating said moving signature vertically and horizontally, and rotating said moving signature, and initializing a divider, computing a initial similarity measure that quantifies the difference between the DOT and MR datasets, deforming said moving signature according to each of said deformation variables, and estimating, for each deformation of said moving signature, the similarity measure between said deformed moving signature and said fixed signature, and incorporating said estimated measure into said registration if said similarity measure has increased.

According to a further aspect of the invention, the method includes multiplying said divider by a multiplication factor, dividing said deformation variables by said divider, and repeating said steps of deforming said moving signature and estimating said similarity measure until said similarity measure converges.

According to a further aspect of the invention, the moving signature is the DOT signature, and said fixed signature is the MR signature.

According to a further aspect of the invention, an estimate of said registration maximizes a similarity measure

$${T}_{P}^{5} = arg {max}_{{T}_{P}^{5}} {S}^{2} ( {Φ}_{P} ( {I}_{f} ) , {Γ}_{{T}_{P}^{5}}^{2} ( {Φ}_{P} ( {I}_{m} ) ) ) ,$$

wherein T_{P}⁵ is a homogenous transformation matrix defined in a plane of projection with 5 degrees of freedom, Φ_{P} is an orthographic projection operator that projects image volume points onto an image plane, P is a 4×4 homogeneous transformation matrix that encodes a principal axis of the orthographic projection, Γ_{T}_{_{P}}_{⁵}² is a mapping operator with translational and rotational degrees of freedom, S² is the similarity metric between 2D projections, and I_{f} and Iₘ are the fixed and moving images, respectively.

According to a further aspect of the invention, the similarity metric for comparing signatures is mutual information, S²=h(I_{I})+h(I_{J})−h(I_{I},I_{J}), wherein I_{I} and I_{J} represent the MR and DOT datasets, h(I) is an entropy of a image intensity I defined as

$$H ( I ) = - {\sum }_{I = L}^{H} {P}_{I} ( I ) log {p}_{I} ( I ) , h ( {I}_{I} , {I}_{J} )$$

is a joint entropy of two image intensities I_{I} and I_{J} defined as

$$H ( {I}_{I} , {I}_{J} ) = - {\sum }_{I = L}^{H} {\sum }_{J = L}^{H} {p}_{{I}_{I} , {I}_{J}} ( I , J ) log {p}_{{I}_{I} , {I}_{J}} ( I , J ) ,$$

I and J are the intensities ranging from lower limit L to higher limit H for I_{I} and I_{J}, respectively, p_{I}_{_{I}}(I) is a probability density function (PDF of image I_{I}, and p_{I}_{_{I}}_{,I}_{_{J}} (I,J) is the joint PDF of images I_{I} and I_{J}, wherein a PDF is represented by a normalized image histogram.

According to a further aspect of the invention, generating said projection signatures and registering said signatures is performed on a graphics processing unit (GPU).

According to another aspect of the invention, there is provided a program storage device readable by a computer, tangibly embodying a program of instructions executable by the computer to perform the method steps for joint analysis of non-concurrent magnetic resonance (MR) and diffuse optical tomography (DOT) images of the breast.

### BRIEF DESCRIPTION OF THE DRAWINGS

FIG. 1 shows an exemplary, non-limiting workflow of an OMIRAD system of an embodiment of the invention.

FIG. 2 depicts an exemplary visualization display showing same patient MRI and DOT (blood volume) datasets before registration.

FIG. 3 depicts a schematic of a DOT instrument, according to an embodiment of the invention.

FIG. 4 shows an example of a 3D distribution of THC (μM) in a patient breast with an invasive ductal carcinoma, according to an embodiment of the invention.

FIGS. 5(a)-(d) illustrate the generation of 2D signatures from 3D volumes, according to an embodiment of the invention.

FIGS. 6(a)-(d) illustrate the different transformation models that can be used in medical image registration, according to an embodiment of the invention.

FIGS. 7(a)-(b) are flowcharts of a registration algorithm according to an embodiment of the invention.

FIGS. 8(a)-(b) illustrate the results of random-walker breast MRI 3D image segmentation, according to an embodiment of the invention.

FIG. 9 is a flowchart of a breast segmentation algorithm according to an embodiment of the invention.

FIGS. 10(a)-(c) depict exemplary compressed breast models, according to an embodiment of the invention.

FIGS. 11(a)-(d) shows the visual results of translations along the Z and X axes, according to an embodiment of the invention.

FIGS. 12(a)-(c) shows examples of rotations applied and the resulting alignments, according to an embodiment of the invention.

FIG. 13 shows an exemplary arrangement of 26 points arranged on the cube and used to compute the target registration error, according to an embodiment of the invention.

FIG. 14 is a graph of the THC distribution in a DOT dataset, according to an embodiment of the invention.

FIG. 15 is a bar graph showing statistical values computed in the registered DOT datasets as well as the difference measures, according to an embodiment of the invention.

FIGS. 16, 17, and 18 show the visual results of the registration algorithm when applied to 3 patients, according to an embodiment of the invention.

FIG. 19 shows the different statistics due to translations of the MR segmentation area inside the THC DOT dataset, according to an embodiment of the invention.

FIG. 20 is a block diagram of an exemplary computer system for implementing a method for combining breast image data obtained at different times, in different geometries and by different techniques according to an embodiment of the invention.

### DETAILED DESCRIPTION OF EXEMPLARY EMBODIMENTS

Exemplary embodiments of the invention as described herein generally include systems and methods for combining breast image data obtained at different times using different geometries and different techniques. Accordingly, while the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the spirit and scope of the invention.

As used herein, the term “image” refers to multi-dimensional data composed of discrete image elements (e.g., pixels for 2-D images and voxels for 3-D images). The image may be, for example, a medical image of a subject collected by computer tomography, magnetic resonance imaging, ultrasound, or any other medical imaging system known to one of skill in the art. The image may also be provided from non-medical contexts, such as, for example, remote sensing systems, electron microscopy, etc. Although an image can be thought of as a function from R³ to R, the methods of the inventions are not limited to such images, and can be applied to images of any dimension, e.g., a 2-D picture or a 3-D volume. For a 2- or 3-dimensional image, the domain of the image is typically a 2- or 3-dimensional rectangular array, wherein each pixel or voxel can be addressed with reference to a set of 2 or 3 mutually orthogonal axes. The terms “digital” and “digitized” as used herein will refer to images or volumes, as appropriate, in a digital or digitized format acquired via a digital acquisition system or via conversion from an analog image.

### Software Platform

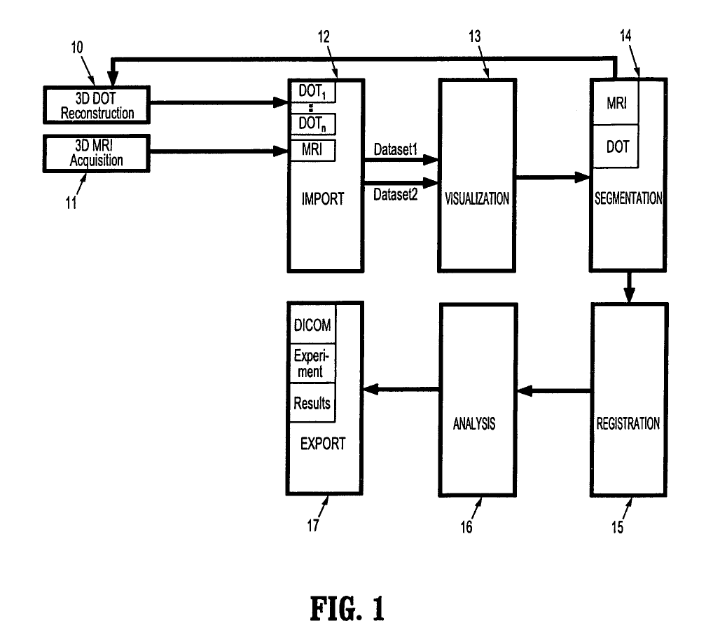

A software system according to an embodiment of the invention for combining non-concurrent MRI and DOT, referred to herein as the Optical & Multimodal Imaging Platform for Research Assessment & Diagnosis (OMIRAD), enables multimodal 3D image visualization and manipulation of datasets based on a variety of 3D rendering techniques, and through simultaneous control of multiple fields of view, streamlines quantitative analyses of structural and functional data. An exemplary, non-limiting workflow of an OMIRAD system of an embodiment of the invention is shown in FIG. 1. Such a system accepts two types of data formats: MRI datasets 11 in the DICOM (Digital Imaging & Communications in Medicine) format widely used for medical images, and DOT datasets 10 in either the TOAST (Time-resolved Optical Absorption and Scattering Tomography) format, developed at University College London, or the NIRFAST (Near Infrared Frequency Domain Absorption and Scatter Tomography) format developed at Dartmouth College. Datasets are converted into a common binary format through a user-friendly interface.

A patient browser in an Import Module 12 allows the user to select any two 3D datasets for visualization and/or registration. The visualization stage 13 permits the user to inspect each dataset, both through volume rendering and multi-planar reformatting (MPR) visualization, and to define a volume of interest (VOI) through morphological operations such as punching. Punching involves determining a 3D region of an object from a 2D region specified on an orthographic projection of the same object. This 3D region can then be removed or retained. This type of operation enables an easy editing of 3D structures. This is a useful stage, as the user removes parts of the data that should not be used in the registration process. The breast MR image segmentation performed by segmentation stage 14 enables a priori structural information derived from MRI to be incorporated into the reconstruction of DOT data 10. The user may decide to roughly align one volume to the other, before starting the automatic registration performed by the registration stage 15. Once the registration is completed, several tools are available to the user in the analysis stage 16 for assessment of the results, including fused synchronized MPR and volume manipulation. These results, along with the image data, are made available for export at the export stage 17.

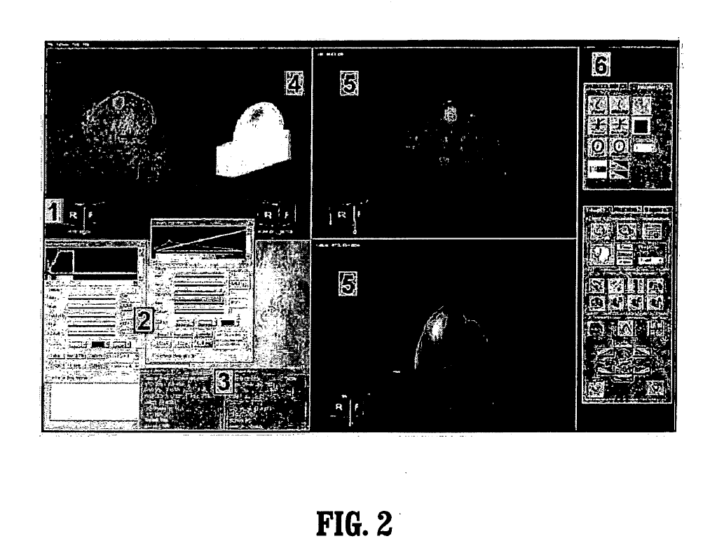

An exemplary visualization display showing same patient MRI and DOT (blood volume) datasets before registration is depicted in FIG. 2. After the appropriate color transfer functions are applied, one can observe the location of the invasive ductal carcinoma diagnosed in this patient breast. The following components are shown from left to right: 1. Orientation cube, 2. Transfer function editors, 3. Data attribute windows, 4. Volume rendering window, 5. MPR windows, 6. Command tabs.

### DOT System Overview

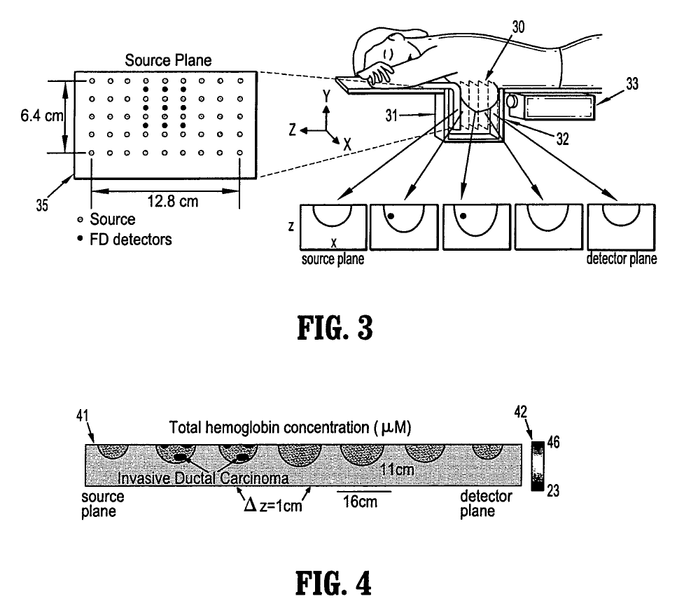

FIG. 3 depicts a schematic of a DOT instrument. A hybrid continuous-wave (CW) and frequency-domain (FD) parallel-plane DOT system has been calibrated for breast cancer imaging using tissue phantoms and normal breast images. As shown in the figure, the breast 30 is typically softly compressed between the source plate 31 and a viewing window 32, to a thickness of about 5.5-7.5 cm. The breast box is filled with a matching fluid, such as Intralipid and Indian ink, that has optical properties similar to human tissue. In this exemplary, non-limiting apparatus, four laser diodes (690, 750, 786, and 830 nm wavelength), amplitude modulated at 70 MHz, are used as light sources, with a grid of 9×5=45 source positions with a spacing of about 1.6 cm, as shown in source plane 35. The dimensions indicated in the figure are exemplary, and source planes of other sizes are within the scope of an embodiment of the invention. For (CW) transmission detection a 24×41=984 grid of pixels is sampled from the CCD camera 33, which corresponds to a detector separation of about 3 mm on the detection window. For remission detection (FD), a 3×3=9 grid (˜1.6 cm spacing) of detector fibers located on the source plate is used. Remission detection is used to determine the average optical properties of the breast. These values are used as an initial guess for the non-linear image reconstruction. The CCD data is used for the image reconstruction. For each source position and wavelength, FD measurements can be obtained via the nine detector fibers on the source plate and CW measurements can be obtained simultaneously via CCD camera in transmission. The amplitude and phase information obtained from the FD measurements are used to quantify bulk optical properties, and the CW transmission data is used to reconstruct a 3D tomography of optical properties within the breast.

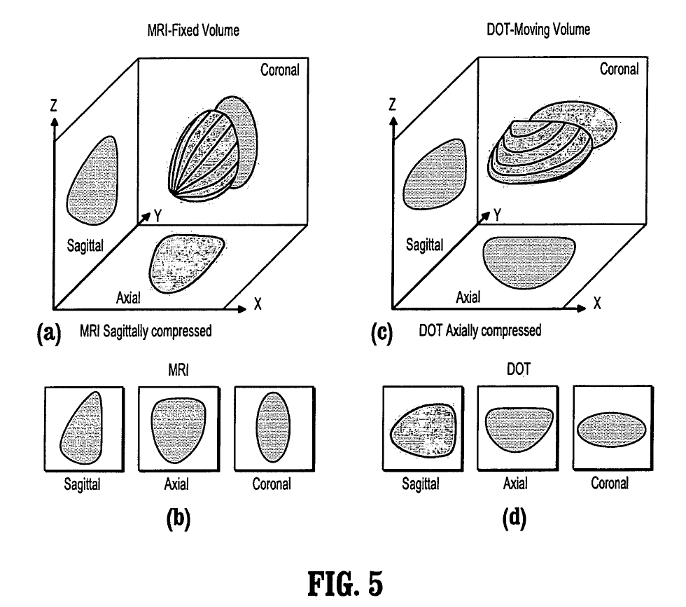

In order to reconstruct the absorption and scattering image, an inverse problem associated with the photon diffusion equation is solved by iterative gradient-based optimization that reconstructs chromophore concentrations (CHb, CHbO₂) and scattering coefficients directly using data from all wavelengths simultaneously. A variation of the open-source software package TOAST (Time-resolved Optical Absorption and Scattering Tomography) can be used for these reconstructions. TOAST determines the optical properties inside a medium by adjusting these parameters such that the difference between the modeled and experimental light measurements at the sample surface is minimized. Images of physiologically relevant variables, such as total hemoglobin concentration (THC), blood oxygenation saturation (StO₂), and scattering are thus obtained. FIG. 4 shows an example of a 3D distribution of THC (μM) in a patient breast with an invasive ductal carcinoma. Consecutive 2D patient slices 41 are adjacent to each other. Each hemisphere represents a successive axial slice of data from the patient's breast from the source plane to the detector plane (note the correspondence with FIG. 3). The Az is the distance from one slice to the next slice, and the 16 cm and 11 cm markings indicate the size of each slice. The bar 42 on the right marked with 23 and 46 represents the scale of total hemoglobin concentration.

The resulting DOT dataset is a finite element (FE) model containing on average 50,000 nodes and 200,000 tetrahedral elements. Each node is associated with the reconstructed physiological values such as THC and StO₂. To facilitate registration of DOT and MR images, the FE model is automatically resampled into a 3D voxelized volume. The smallest bounding box surrounding the FE model is identified, and this volume is divided into voxels (1283 by default). Every voxel is associated to the tetrahedral element to which it belongs and finally, using the element's shape functions, the correct physiological value is interpolated at the location of the voxel.

### 3D/3D DOT to MRI Image Registration

3D-DOT/3D-MRI image registration presents several new challenges, described above. To address these challenges, a registration algorithm should be automatic with little prior user interaction and be robust enough to handle the majority of patient cases. In addition, the process should be computationally efficient for applicability in practice, and yield results useful for combined MRI/DOT analysis.

Consider two datasets to be registered to each other. One dataset is considered the reference and is commonly referred to as the ‘fixed’ dataset. The other dataset is the one onto which the registration transformation is applied. This dataset is commonly referred to as the ‘moving’ dataset. Registration of volumetric data sets (i.e., fixed and moving) involves three steps: first, computation of the similarity measure quantifying a metric for comparing volumes, second, an optimization scheme, which searches through the parameter space (e.g., six dimensional rigid body motion) in order to maximize the similarity measure, and third, a volume warping method, which applies the latest computed set of parameters to the original moving volume to bring it a step closer to the fixed volume.

A registration method according to an embodiment of the invention computes 2D projection images from the two volumes for various projection geometries, and calculates a similarity measure with an optimization scheme which searches through the parameter space. These images are registered within a 2D space, which is a subset of the 3D space of the original registration transformations. Finally, these registrations are performed successively and iteratively in order to estimate all the registration parameters of the original system.

The performance of projection and 2D-2D registration similarity computation is further optimized through the use of graphics processing units (GPU). Multiple two-dimensional signatures (or projections) can represent the volume robustly depending on the way the signatures are generated. An easy way to understand the idea is to derive the motion of an object by looking at three perpendicular shadows of the object.

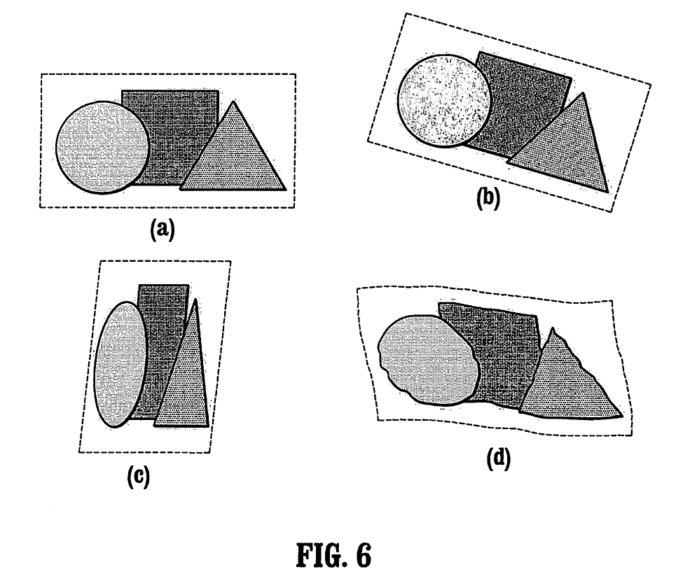

FIGS. 5(a)-(d) illustrate the generation of 2D signatures from 3D volumes. FIGS. 5(a) and (c) respectively depict a sagittally compressed MRI dataset and an axially compressed DOT dataset, with the arrows represent the direction of 2D projections in the three mutually orthogonal directions. FIGS. 5(b) and (d) illustrate the sagittal, axial, and coronal projections for the MRI and DOT datasets, respectively.

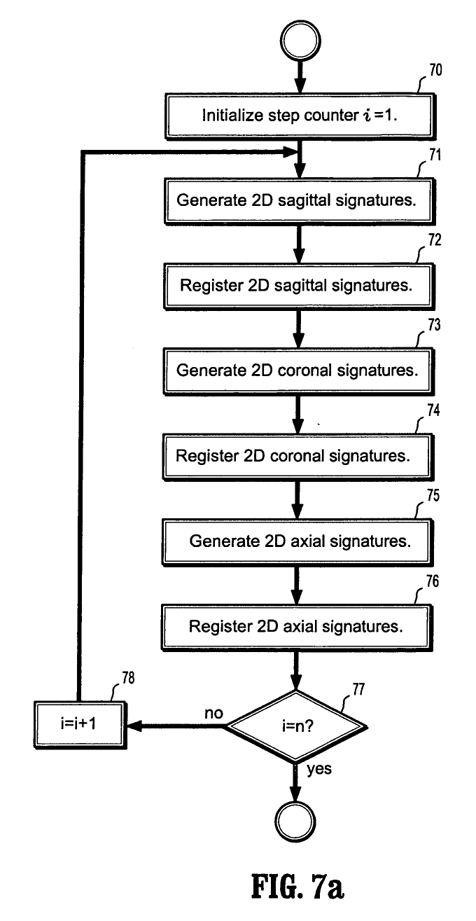

FIGS. 6(a)-(d) illustrate the different transformation models that can be used in medical image registration: rigid, affine, and free-form transformations. FIG. 6(a) shows an original image, and FIG. 6(b) shows the effect of a rigid transformation, which involves only translation and rotation of the original image. Non-rigid registration, depending on complexity, can be classified in two ways: (1) Affine transformations, the effect of which is illustrated in FIG. 6(c), which include non-homogeneous scaling and/or shearing; and (2) free-form transformations, illustrated in FIG. 6(d), which include arbitrary deformations at the voxel level. These transformations can be based on intensity, shape or material properties. The deformations observed across the MR and DOT datasets are due to the difference in compression axis (lateral compression for MR versus axial compression for DOT), and this transformation can be modeled using affine parameters. DOT images do not possess sufficient local structure information for computation of a free-form deformation mapping to register a DOT to an MR dataset.

Given the above challenges, the following parameters can be used in a non-rigid registration algorithm according to an embodiment of the invention. For projection images, a maximum intensity projection (MIP) technique is used. MIP is a computer visualization method for 3D data that projects in the visualization plane those voxels with maximum intensity that fall in the way of parallel rays traced from the viewpoint to the plane of projection. For projection geometries, three mutually orthogonal 2D MIP's are used, in order to achieve greater robustness in the registration algorithm.

A normalized mutual information is used as the similarity measure. Mutual information measures the information that two random variable A and B share. It measures how knowledge of one variable reduces the uncertainty in the other. For example, if A and B are independent, then knowing A does not give any information about B and vice versa, so their normalized mutual information is zero. On the other hand, if A and B are identical then all information given by A is shared with B; therefore knowing A determines the value of B and vice versa, and the normalized mutual information is equal to its maximum possible value of 1. Mutual information quantifies the distance between the joint distribution of A and B from what it would be if A and B were independent. In this case, the moving dataset is deformed until the normalized mutual information between it and the fixed dataset is maximized.

The parameter space includes rigid body motion parameters (translation and rotation), and independent linear scaling in all three dimensions. This results in a 9 dimensional parameter space enabling non-rigid registration: three parameters for translation in x, y and z, three parameters for rotations about three axes, and three parameters for linear scaling in each of the x, y and z directions.

Mathematically, the estimate of the nine degrees-of-freedom (DOF) homogeneous transformation matrix T⁹ is initially given by

$${T}^{9} = arg {max}_{{T}^{9}} {S}^{3} ( {I}_{f} , {Γ}_{{T}^{9}}^{3} ( {I}_{m} ) ) \qquad ( 1 )$$

where Γ_{T}_{⁹}³ is the six DOF (translational and rotational degrees) mapping operator, S³ estimates the similarity metric between two volumes, and I_{f} and Iₘ are the fixed and moving volumetric data, respectively. Both Γ_{T}_{⁹}³ and S³ have a superscript of 3 to indicate that the operations are over three dimensions.

The registration optimization process can be reformulated so it can be applied to each of the two-dimensional signatures, or projections, using the five DOF homogeneous transformation matrix defined in the plane of projection, T_{P}⁵. The five degrees of freedom in the plane of projection correspond to horizontal and vertical translation, horizontal and vertical scaling, and in-plane rotation. The estimate of the transformation matrix is given by:

$${T}_{P}^{5} = arg {max}_{{T}_{P}^{5}} {S}^{2} ( {Φ}_{P} ( {I}_{f} ) , {Γ}_{{T}_{P}^{5}}^{2} ( {Φ}_{P} ( {I}_{m} ) ) ) \qquad ( 2 )$$

where Φ_{P} is an orthographic projection operator, which projects the volume points onto an image plane, P is a 4×4 homogeneous transformation matrix, which encodes the principal axis of the orthographic projection, Γ_{T}_{_{P}}_{⁵}² is a three DOF mapping operator for the translational and rotational degree of freedom, and S² computes the similarity metric between 2D projections. Here Γ_{T}_{_{P}}_{⁵}² and S² have a superscript of 2 to indicate that the operations are over two dimensions.

The similarity metric is mutual information, S²=h(A)+h(B)−h(A,B), where h(x) is the entropy of a random variable x, and h(x,y) is the joint entropy of two random variables x and y, so equation EQ. (2) can be rewritten as:

$${T}_{P}^{5} = arg {max}_{{T}_{P}^{5}} [ h ( A ) + h ( B ) - h ( A , B ) ] , \qquad ( 3 )$$

where A=Φ_{P} (I_{f}) and B=Γ_{T}_{_{P}}_{⁵}² (Φ_{P}(Iₘ)). Entropy is a measure of variability and is defined as h(x)≡−∫p(x)ln p(x)dx, and h(x, y)≡−∫p(x, y)ln p(x, y)dxdy, where p(x) is the probability density function (PDF) of variable x, and p(x,y) is the joint PDF of variables x and y. The entropy h is discretely computed as:

$$\begin{matrix}H ( {I}_{I} ) = - {\sum }_{I = L}^{H} {p}_{{I}_{I}} ( I ) log {p}_{{I}_{I}} ( I ) , \\ H ( {I}_{I} , {I}_{J} ) = - {\sum }_{I = L}^{H} {\sum }_{J = L}^{H} {p}_{{I}_{I} , {I}_{J}} ( I , J ) log {p}_{{I}_{I} , {I}_{J}} ( I , J )\end{matrix} \qquad ( 4 )$$

where I_{I} and I_{J} are two given images, and I and J are the intensities ranging from lower limit L (e.g., 0) to higher limit H (e.g., 255) for I_{I} and I_{J}, respectively. p_{I}_{_{I}} (I) is the PDF of image I_{I}, and p_{I}_{_{I}}_{,I}_{_{J}} (I,J) is the joint PDF of images I_{I} and I_{J}. Here, a PDF is represented by a normalized image histogram.

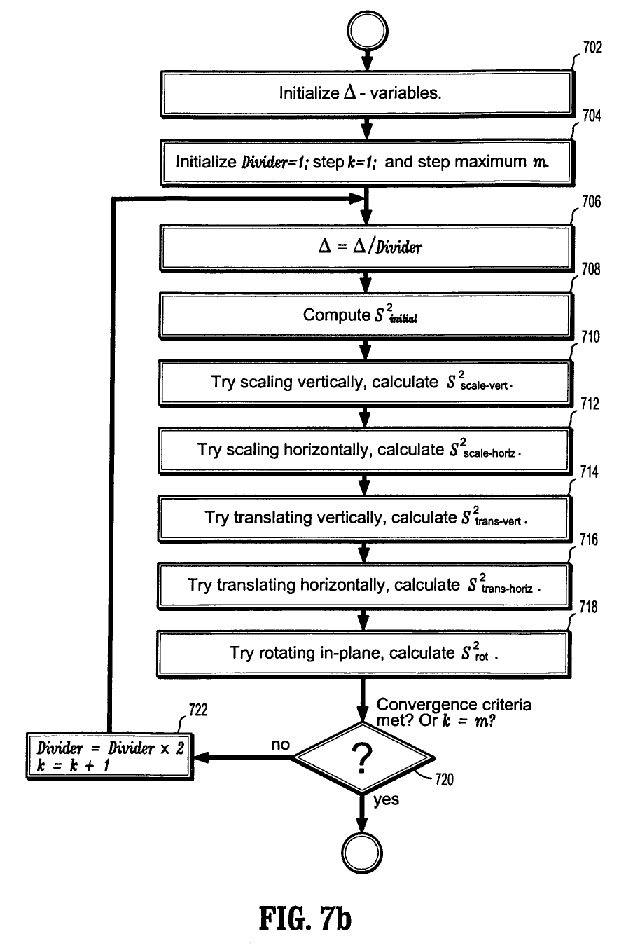

A flowchart of a registration algorithm according to an embodiment of the invention is shown in FIGS. 7(a)-(b). FIG. 7(a) shows the global registration flowchart, while FIG. 7(b) shows the registration of the 2D signatures. For a counter i initialized to 1 at step 70, the three mutually orthogonal 2D signatures (sagittal, coronal and axial) are generated at steps 71, 73, and 75, respectively, for both the fixed and moving volumes for a number of iterations n checked at step 77 (typically n=3). The counter is incremented at step 78. After each 2D signature generation, the moving 2D signature for the projection is registered to the fixed 2D signature at steps 72, 74, and 76 for, respectively, the sagittal, coronal and axial projections. This process is shown schematically in FIG. 7(b), and explained in detail next.

Referring now to FIG. 7(b), at step 702, the Δ variables are initialized. These variables are used to increment/decrement the parameters corresponding to the 5 degrees of freedom, and are initialized as follows: Δscale=Δscale_initial; Δtrans=Δtrans_initial; Δrot=Δrot_initial, where typical initialize values are Δscale_initial=4 mm, Δtrans_initial=4 mm, Δrot_initial=4°. The variable Divider_threshold is a maximum value for Divider, used to update the Δ variables at each iteration. A typical value for Divider_threshold is 40. Then, at step 704, the variable Divider is initialized to 1, step counter k is initialized to 1 and step maximum m is initialized. A typical value for m is 40. At step 706, the Δ variables are updated:

$$Δ scale = \frac{Δ scale}{Divider} ; Δ trans = \frac{Δ trans}{Divider} ; Δ rot = \frac{Δ rot}{Divider} .$$

The initial similarity measure S²ᵢₙᵢₜᵢₐₗ between the two 2D signatures is computed at step 708.

At step 710, the Moving Volume is scaled vertically by ±Δscale, after which S²_{scale-vert} is computed. If there has been an improvement, i.e. S²_{scale-vert}>S²ᵢₙᵢₜᵢₐₗ, then the algorithm proceeds to the next step, otherwise this scaling operation is not applied.

At step 712, the Moving Volume is scaled horizontally by ±Δscale, after which S²_{scale-horiz} is estimated. If there has been an improvement, i.e. S²_{scale-horiz}>S²_{scale-vert}, then the algorithm proceeds to the next step, otherwise this scaling operation is not applied.

At step 714, the Moving Volume is translated vertically by ±Δtrans, after which S²ₜᵣₐₙₛ₋ᵥₑᵣₜ is estimated. If there has been an improvement, i.e. S²ₜᵣₐₙₛ₋ᵥₑᵣₜ>S²_{scale-horiz}, then the algorithm proceeds to the next step, otherwise this scaling operation is not applied.

At step 716, the Moving Volume is translated horizontally by ±Δtrans, after which S²_{trans-horiz} is estimated. If there has been an improvement, i.e. S²_{trans-horiz}>S²ₜᵣₐₙₛ₋ᵥₑᵣₜ, then the algorithm proceeds to the next step, otherwise this scaling operation is not applied.

At step 718, the Moving Volume is rotated in-plane by ±Δrot, after which S²ᵣₒₜ is estimated. If there has been an improvement, i.e. S²ᵣₒₜ>S²_{trans-horiz}, then the algorithm proceeds to the next step, otherwise this scaling operation is not applied.

At step 720, the convergence criteria is checked: if 0<|S²ᵣₒₜ−Sᵢₙₜᵢₐₗ²|≦ΔS² or Divider>Divider_threshold or k=m, then the k-loop is terminated. Here, ΔS² is a pre-determined minimum similarity difference threshold. If no improvements have been made i.e. S²_{reg}=S²ᵢₙᵢₜᵢₐₗ, then the deformation steps are decreased at step 722: Divider=Divider×2, and k is incremented.

### Breast MRI Image Segmentation

A segmentation algorithm according to an embodiment of the invention is based on the random walker algorithm. In this case, the segmentation technique requires little user interaction, and is computationally efficient for practical applications.

This algorithm, based on methods disclosed in “System and Method for Multilabel Random Walker Segmentation Using Prior Models”, U.S. patent application Ser. No. 11/234,965, of Leo Grady, filed on Sep. 26, 2005, and assigned to the same assignee as the present application, and in L. Grady, G. Funka-Lea, “Multi-Label Image Segmentation for Medical Applications Based on Graph-Theoretic Electrical Potentials”, Proceedings of the 8th ECCV04, Workshop on Computer Vision Approaches to Medical Image Analysis and Mathematical Methods in Biomedical Image Analysis, May 15, 2004, Prague, Czech Republic, Springer-Verlag, the contents of both of which are herein incorporated by reference in their entireties, can perform multi-label, semi-automated image segmentation: given a small number of pixels with user-defined labels, one can analytically (and quickly) determine the probability that a random walker starting at each unlabeled pixel will first reach one of the pre-labeled pixels. By assigning each pixel to the label for which the greatest probability is calculated, high-quality image segmentation may be obtained. The segmentation is formulated in a discrete space (i.e., on a graph) using combinatorial analogues of standard operators and principles from continuous potential theory, allowing it to be applied in arbitrary dimensions.

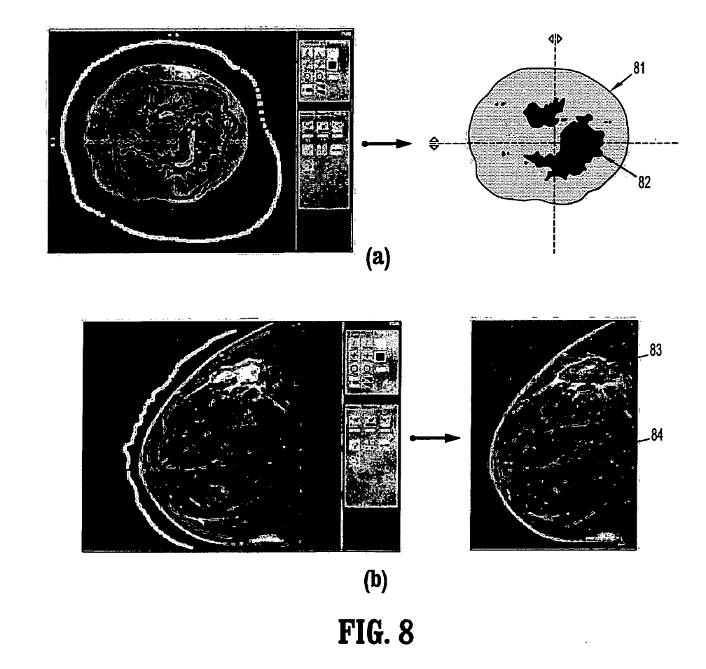

FIGS. 8(a)-(b) illustrate the results of breast MRI 3D image segmentation based on “random walkers” algorithm, according to an embodiment of the invention. FIG. 8(a) depicts segmenting fatty 81 from non-fatty tissue 82, (b) segmenting tumor 83 from non-tumor tissue 84. In each case, the original unsegmented image is shown on the left, and the segmented version is shown on the right. Usually, T₁-weighted MR imaging is performed: these images show lipid as bright, parenchyma as dark, and the tumor also tends to be dark.

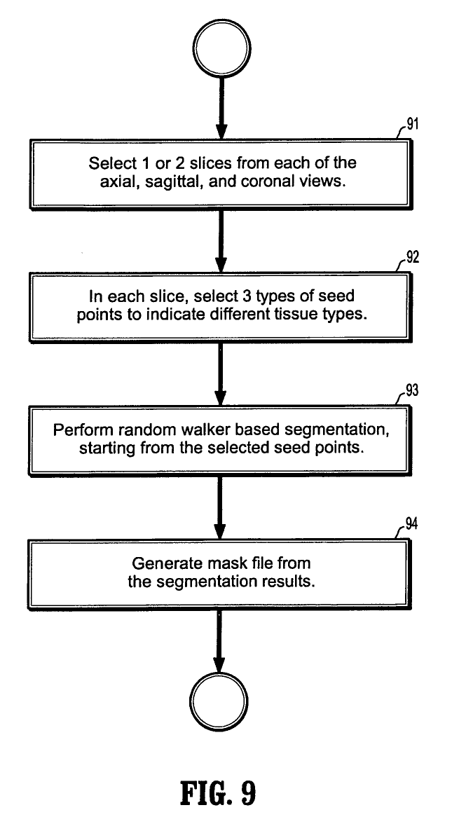

A flowchart of a breast segmentation algorithm according to an embodiment of the invention is shown in FIG. 9. Referring to the figure, at step 91, using a custom-made interactive visual interface, the user scrolls through axial, sagittal, and coronal views of the MRI dataset. In each view, the user selects one or two slices which best incorporate all tissue types. At step 92, the user draws three types of seed points using a virtual ‘brush’ on each of the selected slices, in order to indicate three different tissue types: fatty tissue, non-fatty tissue (parenchyma and/or tumor), and outside the breast tissue. The random walks are performed at step 93. The algorithm generates at step 94 a mask file representing the result of the segmentation. Each voxel in the generated mask is assigned a value (‘fatty’, ‘non-fatty’ or ‘outside’) indicating the type of tissue. The segmented mask file can be finally incorporated at step 95 into a more accurate reconstruction of physiological quantities (such as THC) to generate the DOT dataset.

The algorithm takes two minutes on average for a MRI volume of size 256×256×50, on a Pentium® 4 computer running at 2.0 GHz with 1 gigabyte of RAM.

This algorithm can be used to distinguish fatty from non-fatty tissue and tumor from non-tumor tissue, as shown in FIGS. 8(a)-(b). The MRI segmentation result can be used to isolate the tumor tissue in the image.

One use of spatially registering DOT to MRI data is the ability to treat anatomical information from MRI data as prior information in the DOT chromophore concentration and scattering variables reconstruction process. By segmenting fatty from non-fatty tissue in a MR dataset for example, a priori data can be provided about the tissue which interacts with light in a DOT imaging device. This information can be further incorporated in calculating the inverse associated with the photon diffusion equation, and can lead to a more precise reconstruction of physiological quantities (such as hemoglobin concentration).

### Testing and Results of Registration Using a Simulated Phantom Model

In order to obtain reference results, a method according to an embodiment of the invention was tested using a virtual model of the breast. This model used a hemi-spherical form representing the breast and containing a second sphere of twice the background intensity, representing the tumor. The diameter of the tumor is about 25.6 mm (20% of the spherical form diameter), and the diameter of the spherical form is about 128 mm.

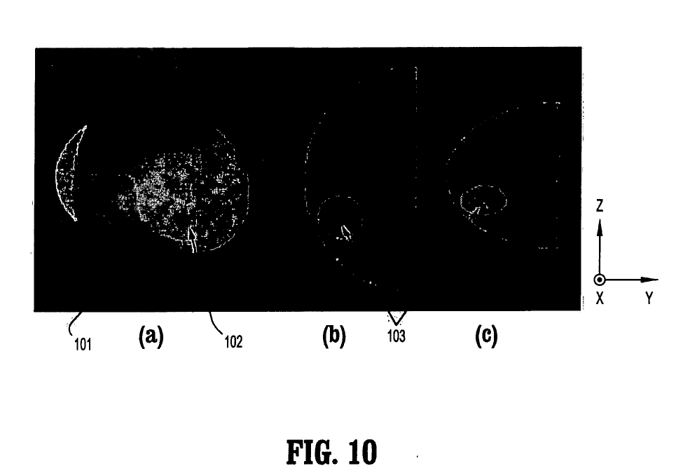

FIGS. 10(a)-(c) depict exemplary compressed models, according to an embodiment of the invention. FIG. 10(a) shows a 3D sagittal perspective view of superimposed MRI 101 and DOT 102 models. FIG. 10(b) shows a sagittal cross-section of MRI model going through the center of the tumor 103, while FIG. 10(c) shows a spatially corresponding sagittal cross-section of DOT model. The coordinate axis are indicated to the right of the figures.

The semi-spherical model is first compressed in the axial direction to simulate the DOT Image. The initial model is again compressed in the sagittal direction to simulate the MR Image. The amount of compression used is about 25% for both the optical and MR images respectively in the axial direction (along the Z axis) and the sagittal direction (along the X axis).

For the axial compression, the z component of the voxel size was decreased by about 25% and the x and y components are proportionally increased to keep the same volume size as the uncompressed model. The sagittal compression is simulated in a similar way by decreasing the x component of the voxel size by about 25%, and the z and y components are proportionally increased to keep the same volume size. The new tumor center position after compression is determined by multiplying the tumor center position in pixels, by the new voxel size.

The experiments described below test a registration algorithm's sensitivity to changes in breast compression, translation and rotation between the MRI and DOT datasets that would be due primarily to patient positioning differences.

### First Set of Simulations: Incremental Translations Along the x, y and z Axes

Initial translations along the x axis are applied incrementally to the DOT (moving) image. The registration was tested after each translation. These translations simulate the difference in patient placement between the two image acquisition processes (translations of +/−˜50 mm). The simulation is repeated with translations applied along the y axis and z axis and the registration is tested for each translation.

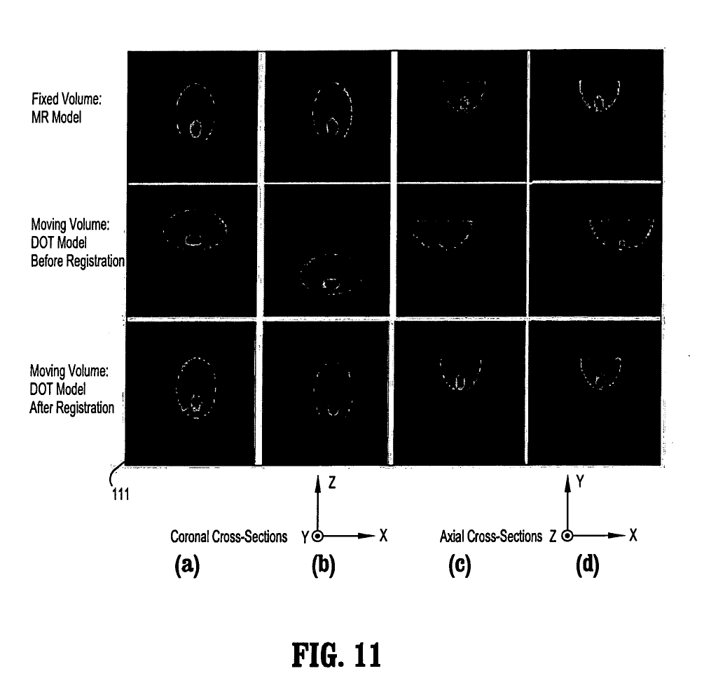

FIGS. 11(a)-(d) shows the visual results of translations along the Z and X axes, with spatially corresponding cross-sections of MRI model in the top row, DOT model before registration in the center row, and the DOT model after registration in the bottom row. The tumor 111 is the smaller, lighter-shaded ellipse, and is only indicated for one of the images for clarity. FIGS. 11(a) and (b) show coronal cross-sections, where the DOT model is translated ±˜50 mm along Z-direction, while FIGS. 11(c) and (d) show axial cross-sections, where the DOT model is translated ±˜50 mm along X-direction. The coordinate axis are indicated to the right of the cross-section labels. The MR model (top row) is the fixed volume in the simulation, and therefore remains unchanged. The DOT model in the center row is the moving volume. This center row shows different initial starting points for the DOT model. In FIGS. 11(c) and (d), the tumor appears very small because the cross-sections shown are spatially corresponding to those of the MR model, and show the edge of the tumor. Note that the bottom row, the DOT model after registration, should look as much as possible like the top row, the MR model.

Second Set of Simulations: Incremental Rotations About the x, y, and z-Axes

Several incremental rotations about the x, y, and z axes, both clockwise and counter-clockwise directions, are applied to the DOT volume, and the registration is tested after each rotation. This is repeated for rotations around the y and z axis, and the registration is tested for each rotation step.

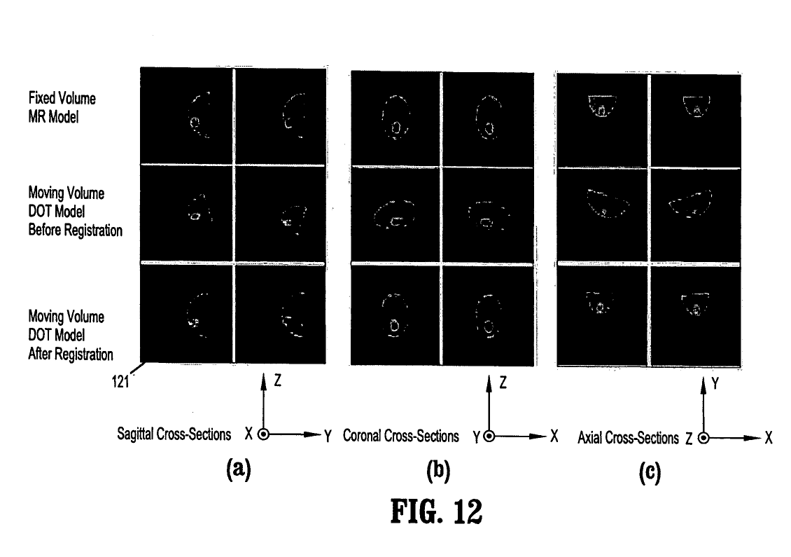

FIGS. 12(a)-(c) shows examples of rotations applied and the resulting alignments for rotations of about +/−18 degrees. The tumor 121 is the smaller, lighter-shaded ellipse, and is only indicated for one of the images for clarity. Spatially corresponding cross-sections are shown of the MRI model in the top row, the DOT model before registration in the center row, and the DOT model after registration in the bottom row. FIG. 12(a) depicts the sagittal cross-sections, where the DOT model is rotated ±18° about the x-axis; FIG. 12(b) shows coronal cross-sections, where the DOT model is rotated 18° about the y-axis; and FIG. 12(c) shows axial cross-sections, where the DOT model is rotated ±18° about the z-axis. The coordinate axis are indicated to the right of the cross-section labels. Here again, the MR model in the top row is the fixed volume in the simulation, and therefore remains unchanged. The DOT model in the center row is the moving volume, and shows different initial rotations for the DOT model. In FIG. 12(c) the tumor appears small because the cross-sections shown spatially correspond to those of the MR model, and show the edge of the tumor. Note that the bottom row, the DOT model after registration, should look as much as possible like the top row, the MR model.

### Third Set of Simulations: Incremental Axial Compression of the Simulated Dot Dataset

Different incremental amounts of compression were applied to the DOT images in the axial direction, along the z axis. To simulate the axial compression, the z component of the voxel size was decreased by about 10% for each test and the x and y components are proportionally increased to keep the same volume size as the uncompressed model. The range of compression used is from 0% compression to 40% compression with a step of 10% for each simulation. Note, no figure is shown in this section.

For most registration tasks, the most significant error measure is the target registration error (TRE), which is the distance after registration between corresponding points not used in calculating the registration transform. The term “target” is used to suggest that the points are typically points within, or on the boundary of lesions. The registration mapping provides the absolute transformation Tᵣₑₛᵤₗₜ that should be applied to the DOT volume in order to be aligned to the MRI volume. This transformation is applied to the tumor center and 26 neighboring points. The points are typically arranged on a cube in which the tumor is inscribed. The cube shares the same center as the tumor.

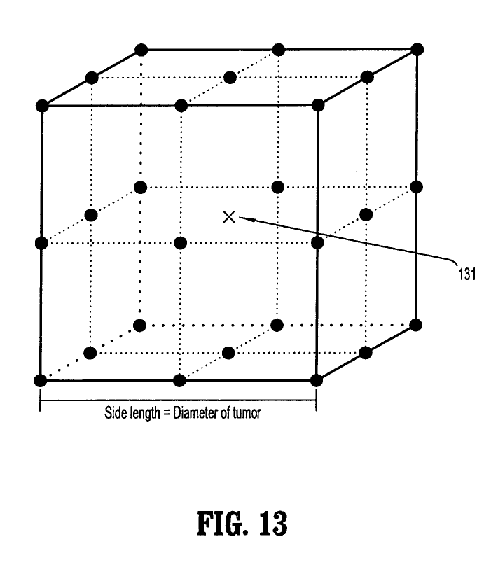

FIG. 13 shows an exemplary arrangement of 26 points arranged on the cube and used to compute the TRE. The tumor is inscribed in the cube, and shares the same center 131 as that of the cube, noted with an ‘x’. This exemplary cube has a side length of about 25.6 mm (equal to the diameter of the tumor). The point positions resulting from the application of the absolute transformation are then compared to the corresponding point positions resulting from the application of the ground truth transformation TGT which provides the expected point positions. This allows determination of the average TRE for each simulation. The TRE is computed as the average Euclidian distance between the 27 pairs of points (P_{GT}ⁱ, Pᵣₑₛᵤₗₜⁱ)

$$TRE = \frac{1}{27} {\sum }_{i = 1}^{27} d ( {P}_{GT} , {P}_{result}^{i} ) \qquad ( 5 )$$

The volume of the tumor after registration is also compared to the tumor before registration and the percentage error is computed. The range of translations chosen during simulations is 40 mm (from −20 to 20 mm) to maintain reasonable simulation parameters. These translations represent typical patient displacements during the image acquisition. Also, the range of rotations chosen is about 36 degrees (from −18 to 18 degrees) for the same reasons.

Table 1, Table 2 and Table 3 show the % volume errors with respect to the original moving volume, and the resulting average Target Registration Errors in mm, as a function of the incremental translation, rotation, and axial compression, respectively. As can be observed, an algorithm according to an embodiment of the invention is more sensitive to rotations than translations, as the error exceeds 5% in some instances. This is explained by the fact that the registration uses 2D signatures of the 3D volume. By applying a rotation to the volume, the shape of the 2D signature changes, whereas by applying a translation the signature is moved compared to the volume of reference while keeping the same form. The change in form due to rotation makes the convergence more challenging. However the larger rotations (more than +10°) will seldom be encountered in reality, where patients usually lie prone in a reproducible manner, and will not cause large rotations. Tests for these larger rotations were conducted in order to explore limitations of the registration technique. For certain points the error rate increases considerably. This is also explained by the use of the 2D signatures. Indeed, when the displacement of the image exceeds the limit of the projector that captures the signature, a part of the information on volume is lost leading to a potential divergence of the registration. Even though the registration transformation is not strictly volume preserving, because of the scaling transformation, the volume percent error shows that within the practical range of deformations, the tumor volume is preserved within an average of about 3% of its original size, which is a reasonable error. Finally, the error due to compression is always under 5%.

TABLE 1

| | Translation along | | Translation along | | Translation along | |
|---|---|---|---|---|---|---|
| | X axis | | Y axis | | Z axis | |
| Translation | | Average | | Average | | Average |
| Amount | Volume | TRE | Volume | TRE | Volume | TRE |
| (mm) | % error | (mm) | % error | (mm) | % error | (mm) |
| −20 | 2.97 | 3.77 | 2.59 | 0.60 | 1.05 | 0.89 |
| −10 | 2.27 | 1.76 | 1.47 | 0.87 | −1.51 | 2.34 |
| 0 | 1.79 | 2.62 | 1.79 | 2.62 | 1.79 | 2.62 |
| 10 | 4.51 | 3.02 | 2.80 | 0.71 | 1.24 | 1.00 |
| 20 | 3.95 | 3.03 | −1.43 | 3.03 | 5.55 | 4.21 |

TABLE 2

| | Rotation about X | | Rotation about Y | | Rotation about Z | |
|---|---|---|---|---|---|---|
| | axis | | axis | | axis | |
| Rotation | | Average | | Average | | Average |
| Amount | Volume | TRE | Volume | TRE | Volume | TRE |
| (degrees) | % error | (mm) | % error | (mm) | % error | (mm) |
| −18 | 7.52 | 7.45 | 2.42 | 2.66 | 3.29 | 10.59 |
| −9 | 10.08 | 11.31 | 0.71 | 0.90 | 7.00 | 4.29 |
| 0 | 1.79 | 2.62 | 1.79 | 2.62 | 1.79 | 2.62 |
| 9 | 2.88 | 2.58 | 4.77 | 2.98 | 5.67 | 1.77 |
| 18 | 0.70 | 0.70 | −0.34 | −0.34 | 4.24 | 4.24 |

TABLE 3

| % Amount of | | |
|---|---|---|
| axial | | Average TRE |
| compression | Volume % error | (mm) |
| 0 | 4.91 | 1.21 |
| 10 | 3.66 | 0.87 |
| 20 | 1.80 | 1.06 |
| 30 | −0.36 | 2.37 |
| 40 | 0.11 | 2.76 |

### Application to Non-Concurrent MRI & DOT Data of Human Subjects

A study involving three patients was performed. This study provides an initial answer to the question of how can functional information on a tumor obtained from DOT data be combined with the anatomical information about the tumor derived from MRI data. Three MRI and three DOT (displaying THC) datasets are used in this experiment.

1. Patient 1: MRI (256×256×22 with 0.63×0.63×4.0 mm pixel size) and mastectomy show an invasive ductal carcinoma of the left breast. The size of the tumor was about 2.1 cm, as measured from pathology.

2. Patient 2: NRI (256×256×60 with 0.7×0.7×1.5 mm pixel size) and biopsy show an invasive ductal carcinoma of the left breast. The size of the tumor was about 5.3 cm, as measured from the MRI (Patient 2 was a neo-adjuvant chemo patient and did not have surgery until later).

3. Patient 3: MRI (512×512×56 with 0.35×0.35×3.93 mm pixel size) and mastectomy show an invasive in-situ carcinoma of the right breast. The size of the tumor was about 2.0 cm, as measured from pathology.

All DOT image acquisitions are similar, and show the patient total hemoglobin concentration (THC). The procedure described in the typical workflow depicted in FIG. 1 was used for visualizing, editing, and registering the MRI and DOT datasets, except that MRI segmentation results were not used to improve the DOT reconstructions.

A quantitative analysis of the resulting data is challenging. According to an embodiment of the invention, a simple analysis method which provides valuable functional information about the carcinoma uses the MRI/DOT registered data to calculate the differences in total hemoglobin concentration (THC) between the volumes inside and outside the segmented tumor, as follows.

1. Segment tumor from non-tumor tissue in the breast MRI dataset, using a segmentation approach according to an embodiment of the invention.

2. Calculate the following statistical quantities from the DOT dataset, within the resulting segmented tumor and non-tumor volumes, taking advantage of the registration of the DOT and MRI datasets: (1) the average THC value over the entire breast: α; (2) the average THC value within the tumor volume defined by the MRI segmentation: β; and (3) the standard deviation of THC for the entire breast: σ.

3. Calculate a new difference measure, defined as the distance from α to β in terms of σ:

$$μ = \frac{( β - α )}{σ} .$$

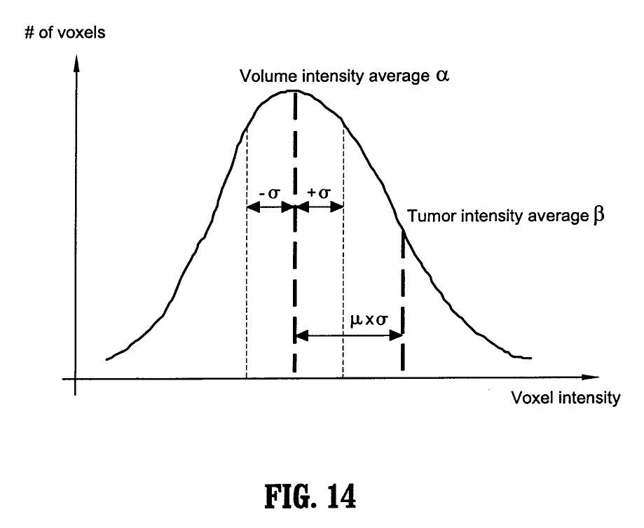

FIG. 14 is a graph of the THC distribution in a DOT dataset as a number of voxels as a function of voxel intensity. The graph also indicates the volume intensity average α, the standard deviation σ, the tumor intensity average β, as well as the new difference measure μ, after DOT-MRI image registration.

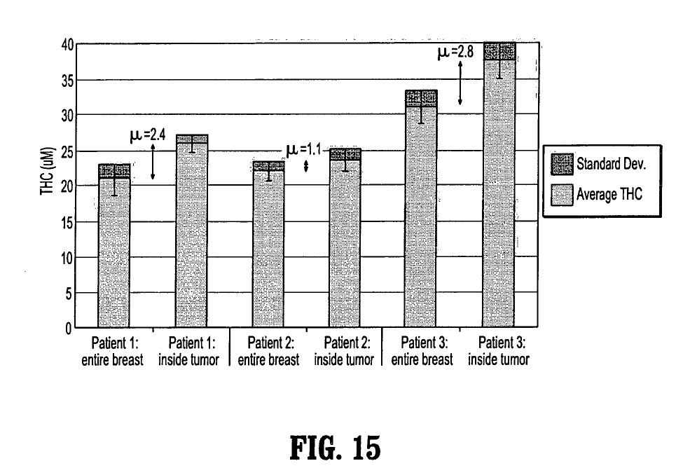

FIG. 15 is a bar graph showing statistical values computed in the registered DOT datasets as well as the difference measures, for each of the 3 patients. Each patient is represented by 2 bars, one for the entire breast, the other for inside the tumor. The segment middle-points are the average THC values (α inside the breast, β inside the tumor); the segment endpoints represent one standard deviation spread σ, and μ is the difference measure, indicated by the double-headed arrow between each pair of bars. All DOT datasets show average tumor THC values that are one to three standard deviations higher than the average breast THC values. The results also show large variability in average breast THC values from one patient to another (varying from about 21 to 31 μM). This justifies the use of the difference measure μ, which defines a normalized quantity allowing inter-patient comparisons. These results confirm that the tumor areas in patient breasts exhibit significantly higher THC than their surroundings.

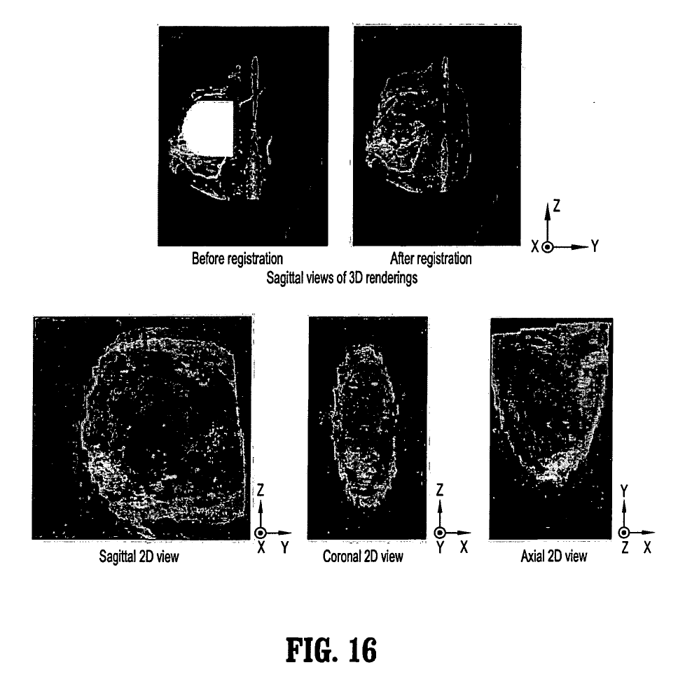

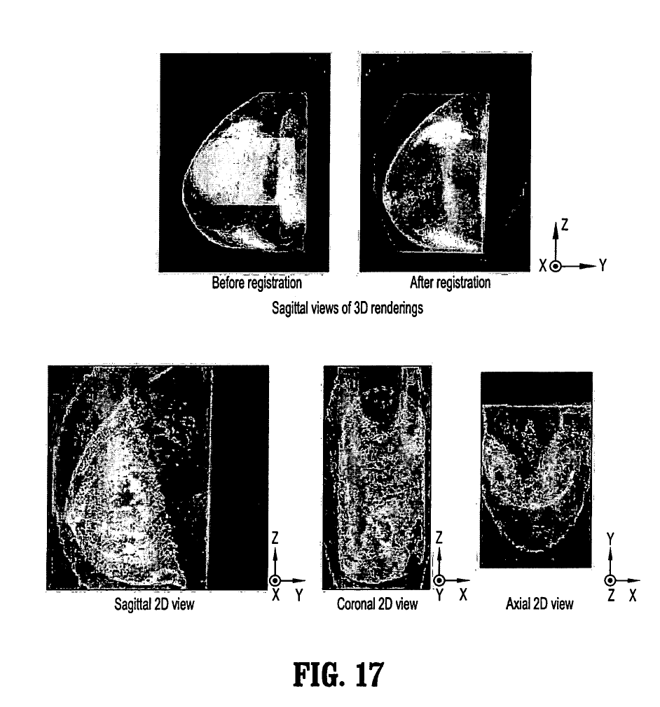

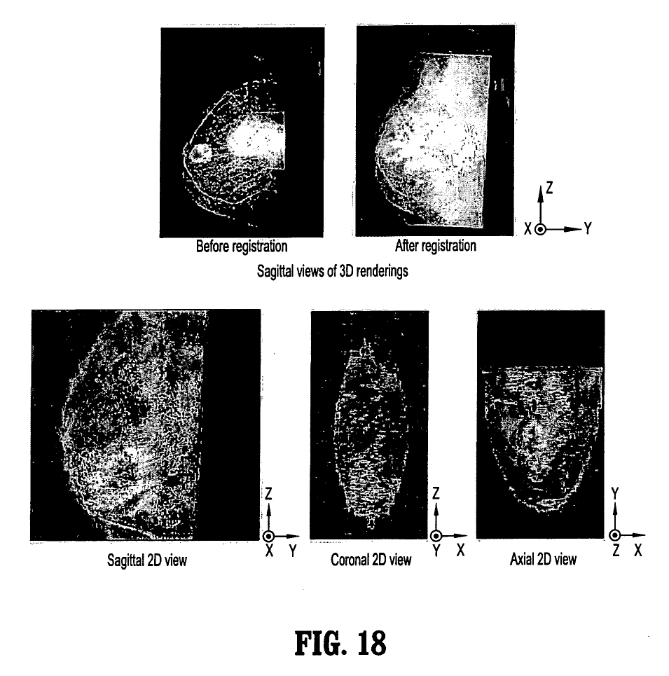

FIGS. 16, 17, and 18 show the visual results of the registration algorithm when applied to patients 1, 2, and 3, respectively. In each figures, the top row shows a sagittal view of superimposed 3D renderings of the MRI and DOT images before and after registration, while the bottom shows the three views of the 2D fused images after registration. The coordinate axis are indicated to the right of the figures. The 2D fused images show the cross-sections going through the center of the tumor. As can be qualitatively ascertained from the figures, registration has improved the alignment of the DOT and MRI datasets. The images also show an overlap between the location of the tumors in the MRI and DOT datasets. Patient 3, shown in FIG. 18, shows particularly good correlation between the two modalities.

The combination of DOT and MR image resolution, the registration technique and the segmentation accuracy in MR all affect the final outcome. Variations in the target registration error (TRE) cause variations in the overlap of the MR segmentation to the THC in the DOT dataset, which in turn cause variations in the quantification of the computed difference measure μ. However because the THC is a slowly varying quantity in the DOT dataset, only small variations are expected in μ.

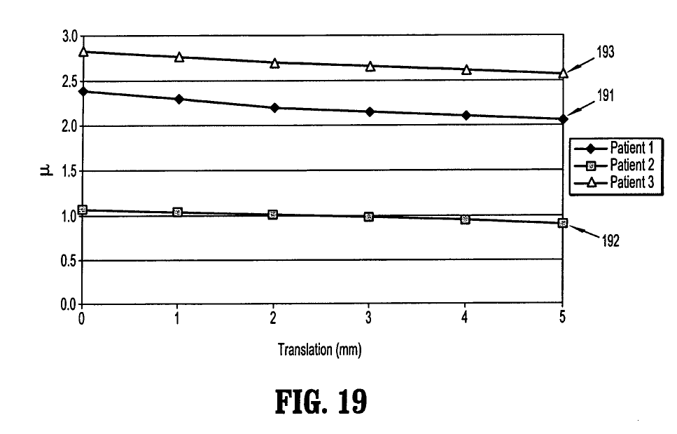

In order to test this hypothesis, variations in the TRE were simulated by translating incrementally the MR segmentation area in the direction of maximum THC gradient in the DOT dataset. This enabled assessment of the upper bound of the quantification error due to TRE variations. The MR segmentation area was translated 1, 2, 3, 4 and 5 mm. Then the different statistics were computed again, and variations in μ for each patient, due to translations of the MR segmentation area inside the THC DOT dataset, are shown in FIG. 19. Patient 1 is represented by graph 191, patient by graph 192, and patient 3 ty graph 193.

As FIG. 19, shows, in all cases the difference measure decreases in amplitude as the translation distance is increased. This shows that the MR segmentation area is translated away from the THC “hotspot” in the DOT datasets. The variations of μ from the baseline (translation=0 mm) in all cases are less than 15% and μ remains equal to or larger than 1, i.e. the average THC inside the segmentation area remains more than one standard deviation away from the overall dataset average THC. Even though these results are limited to only three patients, they exhibit a relative robustness of the registration-segmentation-quantification approach to errors in automatic registration and segmentation. It is also worth noting that these results may apply more generally to patients with breast cancer tumors of sizes within the size range tested, between 2 cm and 5 cm, which is typical.

A co-registration technique according to an embodiment of the invention can be improved by providing additional structural information on the DOT dataset. One way to achieve this goal is to provide a more accurate surface map of the patient's breast as it is scanned in the DOT device, using stereo cameras for example.

### System Implementation

It is to be understood that embodiments of the present invention can be implemented in various forms of hardware, software, firmware, special purpose processes, or a combination thereof. In one embodiment, the present invention can be implemented in software as an application program tangible embodied on a computer readable program storage device. The application program can be uploaded to, and executed by, a machine comprising any suitable architecture.

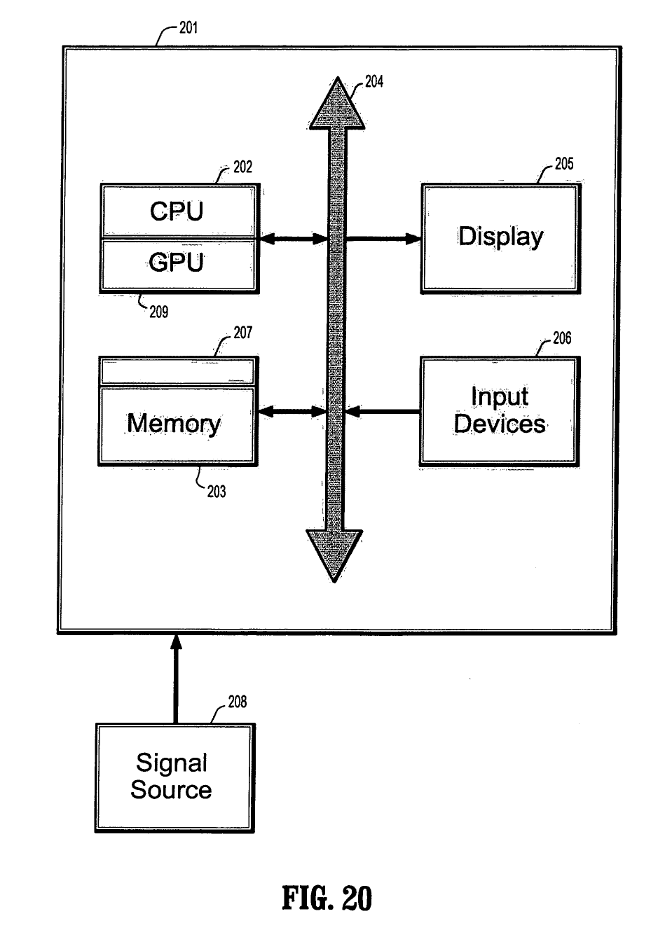

FIG. 20 is a block diagram of an exemplary computer system for implementing a method for combining breast image data obtained at different times, in different geometries and by different techniques according to an embodiment of the invention. Referring now to FIG. 20, a computer system 201 for implementing the present invention can comprise, inter alia, a central processing unit (CPU) 202, a graphics processing unit (GPU) 209, a memory 203 and an input/output (I/O) interface 204. The computer system 201 is generally coupled through the I/O interface 204 to a display 205 and various input devices 206 such as a mouse and a keyboard. The support circuits can include circuits such as cache, power supplies, clock circuits, and a communication bus. The memory 203 can include random access memory (RAM), read only memory (ROM), disk drive, tape drive, etc., or a combinations thereof. The present invention can be implemented as a routine 207 that is stored in memory 203 and executed by the CPU 202 and/or GPU 209 to process the signal from the signal source 208. As such, the computer system 201 is a general purpose computer system that becomes a specific purpose computer system when executing the routine 207 of the present invention.

The computer system 201 also includes an operating system and micro instruction code. The various processes and functions described herein can either be part of the micro instruction code or part of the application program (or combination thereof) which is executed via the operating system. In addition, various other peripheral devices can be connected to the computer platform such as an additional data storage device and a printing device.

It is to be further understood that, because some of the constituent system components and method steps depicted in the accompanying figures can be implemented in software, the actual connections between the systems components (or the process steps) may differ depending upon the manner in which the present invention is programmed. Given the teachings of the present invention provided herein, one of ordinary skill in the related art will be able to contemplate these and similar implementations or configurations of the present invention.

While the present invention has been described in detail with reference to a preferred embodiment, those skilled in the art will appreciate that various modifications and substitutions can be made thereto without departing from the spirit and scope of the invention as set forth in the appended claims.

What is claimed is:

1. A method for joint analysis of non-concurrent magnetic resonance (MR) and diffuse optical tomography (DOT) images of the breast, comprising the steps of:
providing a digitized MR breast image volume comprising a plurality of intensities corresponding to a 3-dimensional (3D) grid of voxels;
providing a digitized DOT breast dataset comprising a plurality of physiological values corresponding to a finite set of points;
segmenting said breast MR image volume to separate tumorous tissue from non-tumorous tissue;
registering said DOT breast dataset and said MR image volume; and
fusing said registered DOT and MR datasets, wherein said fused dataset is adapted for analysis.

2. The method of claim 1, wherein said physiological values include one or more of total hemoglobin concentration, blood oxygenation saturation, and light scattering data.

3. The method of claim 1, wherein segmenting said breast MR image volume comprises:
selecting at least one axial, at least one coronal, and at least one sagittal slice of said MR image volume;
selecting 3 different seed points in each selected slice, said seed points representative of fatty breast tissue, non-fatty breast tissue, and non-breast tissue;
determining a probability that a random walker starting at an unselected point reaches one of said selected seed points; and
labeling each unselected point according to the seed point with a highest probability to create a mask file, wherein each point in each slice is labeled as fatty breast tissue, non-fatty breast tissue, or non-breast tissue.

4. The method of claim 3, further comprising resampling said DOT dataset into a 3D volume of voxels corresponding to said MR grid of voxels.

5. The method of claim 4, comprising incorporating said mask file into said DOT dataset.

6. The method of claim 1, wherein registering said DOT breast dataset to said MR image volume comprises:
generating a 2D sagittal projection signature from said MR image and from said DOT dataset;
registering said DOT sagittal signature and said MR sagittal signature;
generating a 2D coronal projection signature from said MR image and from said DOT dataset;
registering said DOT coronal signature and said MR coronal signature;
generating a 2D axial projection signature from said MR image and from said DOT dataset;
registering said DOT axial signature and said MR axial signature, wherein said 3D registration mapping is defined in terms of said 2D sagittal, coronal, and axial registrations.

7. The method of claim 6, wherein said steps of generating a 2D projection signature and registering said signatures for each of said sagittal, coronal, and axial projections is repeated for a predetermined number of iterations.

8. The method of claim 6, wherein said 2D projection signatures are generated from a maximum intensity projection.

9. The method of claim 6, wherein one of said DOT and MR signatures is a moving signature and the other is a fixed signature, and wherein registering a DOT signature and an MR signature comprises:
initializing deformation variables for scaling said moving signature vertically and horizontally, translating said moving signature vertically and horizontally, and rotating said moving signature, and initializing a divider;
computing a initial similarity measure that quantifies the difference between the DOT and MR datasets;
deforming said moving signature according to each of said deformation variables; and
estimating, for each deformation of said moving signature, the similarity measure between said deformed moving signature and said fixed signature, and incorporating said estimated measure into said registration if said similarity measure has increased.

10. The method of claim 9, further comprising multiplying said divider by a multiplication factor, dividing said deformation variables by said divider, and repeating said steps of deforming said moving signature and estimating said similarity measure until said similarity measure converges.

11. The method of claim 9, wherein said moving signature is the DOT signature, and said fixed signature is the MR signature.

12. The method of claim 9, wherein an estimate of said registration maximizes a similarity measure
$${T}_{P}^{5} = arg {max}_{{T}_{P}^{5}} {S}^{2} ( {Φ}_{P} ( {I}_{f} ) , {Γ}_{{T}_{P}^{5}}^{2} ( {Φ}_{P} ( {I}_{m} ) ) ) ,$$
wherein T_{P}⁵ is a homogenous transformation matrix defined in a plane of projection with 5 degrees of freedom, Φ_{P} is an orthographic projection operator that projects image volume points onto an image plane, P is a 4×4 homogeneous transformation matrix that encodes a principal axis of the orthographic projection, Γ_{T}_{_{P}}_{⁵}² is a mapping operator with translational and rotational degrees of freedom, S² is the similarity metric between 2D projections, and I_{f} and Iₘ are the fixed and moving images, respectively.

13. The method of claim 12, wherein the similarity metric for comparing signatures is mutual information, S²=h(I_{I})+h(I_{J})−h(I_{I},I_{J}), wherein I_{I} and I_{J} represent the MR and DOT datasets, h(I) is an entropy of a image intensity I defined as
$$H ( I ) = - {\sum }_{I = L}^{H} {p}_{I} ( I ) log {p}_{I} ( I ) , h ( {I}_{I} , {I}_{J} )$$
is a joint entropy of two image intensities I_{I} and I_{J} defined as
$$H ( {I}_{I} , {I}_{J} ) = - {\sum }_{I = L}^{H} {\sum }_{J = L}^{H} {p}_{{I}_{I} , {I}_{J}} ( I , J ) log {p}_{{I}_{I} , {I}_{J}} ( I , J ) ,$$
I and J are the intensities ranging from lower limit L to higher limit H for I_{I} and I_{J}, respectively, p_{I}_{_{I}} (I) is a probability density function (PDF of image I_{I}, and p_{I}_{_{I}}_{,I}_{_{J}} (I,J) is the joint PDF of images I_{I} and I_{J}, wherein a PDF is represented by a normalized image histogram.

14. The method of claim 6, wherein generating said projection signatures and registering said signatures is performed on a graphics processing unit (GPU).

15. A method for joint analysis of non-concurrent magnetic resonance (MR) and diffuse optical tomography (DOT) images of the breast, comprising the steps of:
providing a digitized MR breast image volume dataset comprising a plurality of intensities corresponding to a 3-dimensional (3D) grid of points;
providing a digitized DOT breast dataset comprising a plurality of physiological values corresponding to a finite set of points;
computing 2D projection images from said DOT and MR datasets for a plurality of projection geometries,
calculating a similarity measure for each pair of DOT and MR 2D projection images to estimate a transformation that registers said DOT projection to said MR projection; and
repeating said 2D registrations to estimate a 3D registration parameters of said DOT and MR datasets, wherein said registered DOT and MR datasets are adapted for a joint analysis.

16. The method of claim 15, further comprising:
segmenting said breast MR image volume to separate tumorous tissue from non-tumorous tissue;
creating a mask file labeling each point as fatty breast tissue, non-fatty breast tissue, or non-breast tissue;
resampling said DOT dataset into a 3D volume of voxels corresponding to said MR grid of voxels, wherein said mask file is incorporated into said DOT dataset, and fusing said registered DOT and MR datasets.

17. The method of claim 15, wherein said projection geometries comprise a 2D sagittal projection from each dataset, a 2D coronal projection from each dataset, and a 2D axial projection from each dataset, wherein said projections are computed from a maximum intensity projection.

18. The method of claim 15, wherein calculating a similarity measure for each pair of 2D projections comprises:
initializing deformation variables for scaling said DOT projection vertically and horizontally, translating said DOT projection vertically and horizontally, and rotating said DOT projection, and initializing a divider;
computing a initial similarity measure that quantifies the difference between the DOT and MR projections;
deforming said DOT projection according to each of said deformation variables;
estimating, for each deformation of said DOT projection, the similarity measure between said deformed DOT projection and said MR projection, and incorporating said estimated measure into said registration if said similarity measure has increased;
multiplying said divider by a multiplication factor and dividing said deformation variables by said divider; and
repeating said steps of deforming said DOT projection and estimating said similarity measure until said similarity measure converges.

19. A program storage device readable by a computer, tangibly embodying a program of instructions executable by the computer to perform the method steps for joint analysis of non-concurrent magnetic resonance (MR) and diffuse optical tomography (DOT) images of the breast, said method comprising the steps of:
providing a digitized MR breast image volume comprising a plurality of intensities corresponding to a 3-dimensional (3D) grid of voxels;
providing a digitized DOT breast dataset comprising a plurality of physiological values corresponding to a finite set of points;
segmenting said breast MR image volume to separate tumorous tissue from non-tumorous tissue;
registering said DOT breast dataset and said MR image volume; and
fusing said registered DOT and MR datasets, wherein said fused dataset is adapted for analysis.

20. The computer readable program storage device of claim 19, wherein said physiological values include one or more of total hemoglobin concentration, blood oxygenation saturation, and light scattering data.

21. The computer readable program storage device of claim 19, wherein segmenting said breast MR image volume comprises:
selecting at least one axial, at least one coronal, and at least one sagittal slice of said MR image volume;
selecting 3 different seed points in each selected slice, said seed points representative of fatty breast tissue, non-fatty breast tissue, and non-breast tissue;
determining a probability that a random walker starting at an unselected point reaches one of said selected seed points; and
labeling each unselected point according to the seed point with a highest probability to create a mask file, wherein each point in each slice is labeled as fatty breast tissue, non-fatty breast tissue, or non-breast tissue.

22. The computer readable program storage device of claim 21, the method further comprising resampling said DOT dataset into a 3D volume of voxels corresponding to said MR grid of voxels.

23. The computer readable program storage device of claim 22, the method further comprising incorporating said mask file into said DOT dataset.

24. The computer readable program storage device of claim 19, wherein registering said DOT breast dataset to said MR image volume comprises:
generating a 2D sagittal projection signature from said MR image and from said DOT dataset;
registering said DOT sagittal signature and said MR sagittal signature;
generating a 2D coronal projection signature from said MR image and from said DOT dataset;
registering said DOT coronal signature and said MR coronal signature;
generating a 2D axial projection signature from said MR image and from said DOT dataset;
registering said DOT axial signature and said MR axial signature, wherein said 3D registration mapping is defined in terms of said 2D sagittal, coronal, and axial registrations.

25. The computer readable program storage device of claim 24, wherein said steps of generating a 2D projection signature and registering said signatures for each of said sagittal, coronal, and axial projections is repeated for a pre-determined number of iterations.

26. The computer readable program storage device of claim 24, wherein said 2D projection signatures are generated from a maximum intensity projection.

27. The computer readable program storage device of claim 24, wherein one of said DOT and MR signatures is a moving signature and the other is a fixed signature, and wherein registering a DOT signature and an MR signature comprises:
initializing deformation variables for scaling said moving signature vertically and horizontally, translating said moving signature vertically and horizontally, and rotating said moving signature, and initializing a divider;
computing a initial similarity measure that quantifies the difference between the DOT and MR datasets;
deforming said moving signature according to each of said deformation variables; and
estimating, for each deformation of said moving signature, the similarity measure between said deformed moving signature and said fixed signature, and incorporating said estimated measure into said registration if said similarity measure has increased.

28. The computer readable program storage device of claim 27, the method further comprising multiplying said divider by a multiplication factor, dividing said deformation variables by said divider, and repeating said steps of deforming said moving signature and estimating said similarity measure until said similarity measure converges.

29. The computer readable program storage device of claim 27, wherein said moving signature is the DOT signature, and said fixed signature is the MR signature.

30. The computer readable program storage device of claim 27, wherein an estimate of said registration maximizes a similarity measure
$${T}_{P}^{5} = arg {max}_{{T}_{P}^{5}} {S}^{2} ( {Φ}_{P} ( {I}_{f} ) , {Γ}_{{T}_{P}^{5}}^{2} ( {Φ}_{P} ( {I}_{m} ) ) ) ,$$
wherein T_{P}⁵ is a homogenous transformation matrix defined in a plane of projection with 5 degrees of freedom, Φ_{P} is an orthographic projection operator that projects image volume points onto an image plane, P is a 4×4 homogeneous transformation matrix that encodes a principal axis of the orthographic projection, Γ_{T}_{⁵}² is a mapping operator with translational and rotational degrees of freedom, S² is the similarity metric between 2D projections, and I_{f} and Iₘ are the fixed and moving images, respectively.

31. The computer readable program storage device of claim 30, wherein the similarity metric for comparing signatures is mutual information, S²=h(I_{I})+h(I_{J})−h(I_{I},I_{J}), wherein I_{I} and I_{J} represent the MR and DOT datasets, h(I) is an entropy of a image intensity I defined as
$$H ( I ) = - {\sum }_{I = L}^{H} {p}_{I} ( I ) log {p}_{I} ( I ) , h ( {I}_{I} , {I}_{J} )$$
is a joint entropy of two image intensities I_{I} and I_{J} defined as
$$H ( {I}_{I} , {I}_{J} ) = - {\sum }_{I = L}^{H} {\sum }_{J = L}^{H} {p}_{{I}_{I} , {I}_{J}} ( I , J ) log {p}_{{I}_{I} , {I}_{J}} ( I , J ) ,$$
I and J are the intensities ranging from lower limit L to higher limit H for I_{I} and I_{J}, respectively, p_{I}_{_{I}} (I) is a probability density function (PDF of image I_{I}, and p_{I}_{_{I}}_{,I}_{_{J}} (I,J) is the joint PDF of images I_{I} and I_{J}, wherein a PDF is represented by a normalized image histogram.

32. The computer readable program storage device of claim 24, wherein generating said projection signatures and registering said signatures is performed on a graphics processing unit (GPU).
